(19) United States
(12) Patent Application Publication
(10) Pub. No.: US 2006/0183964 A1
(43) Pub. Date: Aug. 17, 2006

(54) Device for self-monitoring of vocal intensity

(76) Inventor: Thomas David Kehoe, Boulder, CO (US)

Correspondence Address: Thomas David Kehoe, 720 31st St., Boulder, CO, 80303-2402, US

(21) Appl. No.: 11/060,003

(22) Filed: Feb. 17, 2005

(51) Int. Cl. A61F 5/58

(52) U.S. Cl. 600/23

(57) ABSTRACT

A device to monitor vocal information such as vocal volume and frequency and provide such information in a plurality of forms, such as tactile, audible, temporal, and visual; and to induce altered vocal volume and/or frequency by altering how the user hears his or her voice, such as by shifting the frequency at which the user hears his or her own voice; for the treatment of vocal nodules, Parkinson's disease, or stuttering.

### FIELD OF THE INVENTION

The present invention relates to a speech training and monitoring apparatus.

### BACKGROUND OF THE INVENTION

Certain individuals develop vocal nodules as a result of excessive vocal intensity. These nodules on the individual's vocal folds (also known as vocal cords) begin as a reddening, then thicken, swell, turn gray and, in the last stage, become fibrotic and callous-like.

Most typically, children three to five years old develop vocal nodules as a result of yelling or screaming. In adults, the disorder can be caused by speaking, singing, or yelling (such as cheerleading) for long periods of time.

Non-medical treatment consists of a speech-language pathologist identifying the behaviors causing the vocal nodules, and then teaching the individual to avoid such behaviors. Individuals who fail to modify their behavior may, as a last resort, have to resort to surgery.

In contrast to too-intense vocal fold abuse, the opposite can occur in persons with Parkinson's Disease. A common symptom of the speech of individuals with Parkinson's Disease is a vocal intensity that is too low. Speech therapy is employed to teach an individual to raise their intensity level. It has been shown that Parkinson's patients have difficulty self-monitoring their voice intensity.

In U.S. Pat. No. 5,015,179 (issued May 14, 1991), Joseph A. Resnick teaches a device and method of speech training that monitors the volume or intensity of a user's voice and displays this volume as a row of five green, yellow and red lights. The inventor suggests that his device is useful “for hard of hearing and/or deaf persons [as well as] those with pitch disorders, vocal nodules, and inappropriate volume of speech (either too loud or too soft, such as seen in many Parkinson's and dysarthric patients).”

While such a device can be effective with adults, children are less likely to pay attention to green or red lights. The need exists for a device that alerts the user in other, more compelling, and/or more rewarding ways.

While vocal volume intensity is the primary issue in the development of vocal nodules, vocal frequency or pitch is also an issue. A variety of patents teach devices that monitor vocal frequency and display such information visually (U.S. Pat. No. 3,881,059, issued April 1975; U.S. Pat. No. 4,580,133, issued Apr. 1, 1986; U.S. Pat. No. 4,641,343, issued Feb. 3, 1987). However, none of these inventions provide information to the user in non-visual ways

Each of the above noted methods and systems provide relief for individuals who suffer from vocal nodules. Due to the limitations associated with each system, it has been determined, therefore, that a need exists for vocal intensity device and method of treatment that monitors such intensity that provides effective, behavior-changing feedback to the user.

### REFERENCES CITED

U.S. Patent Documents

U.S. Pat. No. 3,881,059; Stewart; April 1975; 704/276.
U.S. Pat. No. 4,580,133; Matsuoka et al; Apr. 1, 1986; 434/185.
U.S. Pat. No. 4,641,343; Holland et al; Feb. 3, 1987; 381/48.
U.S. Pat. No. 5,015,179; Resnick; May 14, 1991; 434/185.

### BRIEF SUMMARY OF THE INVENTION

Generally speaking, in accordance with the Invention, a device that monitors vocal intensity and/or frequency and presents audible, tactile, temporal, and/or visual information is provided.

An embodiment of the Invention uses a contact microphone, such as a throat microphone. Another embodiment of the Invention uses non-contact microphone, such as a lapel microphone.

An embodiment of the Invention analyzes the user's vocal volume or intensity. Another embodiment of the Invention analyzes the user's vocal frequency or pitch.

An embodiment of the Invention alerts the user when his or her vocal volume and/or frequency is too high. Another embodiment of the Invention alerts the user when his or her vocal volume and/or frequency is too low.

An embodiment of the Invention alerts the user tactilely such as with a vibrating motor. Another embodiment of the Invention alerts the user audibly such as with red or green lights. Yet another embodiment of the Invention alerts the user audibly such as with a tone in earphones.

An embodiment of the Invention measures and displays the time that the user's voice exceeds target thresholds (e.g., is too loud). Another embodiment of the Inventions measures and displays the time that the user's voice fails below target thresholds (e.g., is too quiet). Another embodiment of the Invention provides a second clock to measure and display the time the device is switched on.

An embodiment of the Invention induces target behavior such as a low vocal pitch by altering the pitch at which the user hears his or her voice.

Accordingly, it is an object of the invention to prevent or reduce vocal nodules.

It is another object of the invention to improve the intelligibility of the speech of persons with Parkinson's disease.

It is another object of the invention to improve the intelligibility of the speech of persons who are deaf or hard of hearing.

It is another object of the invention to enable individuals with inherently soft voices increase their intelligibility by teaching them to speak up.

Still other objects and advantages of the invention will, in part, be obvious and will, in part, be apparent from the specification.

The invention accordingly comprises the features of construction, combinations of elements and arrangements of parts which will be exemplified in the constructions hereinafter set forth, and the scope of the invention will be indicated in the claims.

### DETAILED DESCRIPTION OF THE PREFERRED EMBODIMENTS

In the first configuration of the Invention, a simplified device is made which analyzes vocal intensity (volume or amplitude) and vocal frequency, then presents this information as visual, tactile, and auditory feedback.

The user wears a throat microphone. Such microphones are made by Enhanced Listening Technologies, of West Palm Beach, Fla.; and Ceotronics, of Germany.

The microphone plugs into 3.5 mm jack. A resistor provides bias power to the microphone. A potentiometer provides microphone gain control, which sets the threshold level at which the feedback switches on or off.

An op-amp (a LM6042, made by National Semiconductor) amplifies and filters the audio signal.

A frequency-to-voltage converter (a LM2907, made by National Semiconductor) analyzes the audio amplitude and frequency, converting this into a DC signal. Alternatively a rectifier can be used, to analyze amplitude without frequency. A rectifier consists of two diodes, and a resistor and capacitor to dampen the DC signal.

The DC signal then goes to a comparator. A quad comparator (LP339, made by National Semiconductor) is used. The first comparator compares the DC signal to a fixed DC reference voltage. Two resistors set the reference voltage, typically half of the circuit voltage.

The output of the first comparator switches on a red LED light, visually indicating that the user's voice has exceeded the threshold. This output signal also goes to the input of the second comparator. The second comparator is set up as an inverter. The output of the second comparator drives a green LED light. Thus a single bicolor LED displays as green when the user's vocal intensity is below the threshold, and switches to red when the user's vocal intensity exceeds the threshold.

The output of the second comparator also goes to the input of the third comparator. This non-inverting comparator provides a higher current output for the vibrating motor. Vibrating motors made by JinLong Machinery are preferred, such as the 6SH1-0671A. This provides tactile feedback when the user's vocal intensity exceeds a threshold.

The fourth comparator is set up to synthesize a square wave at approximately 125 Hz. This square wave is then smoothed by a group of resistors and capacitors into a pseudo-sine wave. This provides a pleasant tone. If a harsh sound is preferred as aversive stimulation, decreasing the value a capacitor increasing the frequency; or decreasing the value of another capacitor reduces the smoothing of the signal.

A switch enables the user to switch between hearing the tone or his/her voice. A power amplifier (a LM4881, made by National Semiconductor) provides this auditory feedback to a 3.5 mm earphone jack. A potentiometer provides a volume control.

A voltage regulator (LP2982, made by National Semiconductor) provides the power supply. A 9-volt battery is used for power, which is converted to five volts to run the circuit. To increase battery life, the circuit can also run at four volts.

The entire circuit easily fits into a small case similar to a pager, which clips onto the user's belt. The user may wear earphones to receive the auditory feedback. Alternatively the user may forego wearing earphones and rely instead of the tactile and/or visual feedback.

In a second embodiment of the invention, a frequency shifting computer chip, such as a YSS222-D, made by Yamaha Semiconductor, can be used to shift the user's vocal signal higher or lower in pitch. When this chip is inserted into the circuit, the user can hear his or her voice sounding more relaxed or more tense. This altered auditory feedback can induce tense or relaxed vocal folds, thus actively inducing quieter, more relaxed speech (or louder, tensor speech), with little or no effort on the part of the user.

In a third embodiment of the invention, a double-pole, double-throw switch is inserted between the frequency-to-voltage converter and the first comparator. This switch enables reverses all outputs, so that low vocal intensity is bad instead of good, or vice versa: E.g., the green light that indicates low vocal intensity is switched so that the red light indicates low vocal intensity.

In a fourth embodiment of the invention, one or two LCD clock modules are added. A Red Lion MDMU0000 LCD module is preferred. By connecting pin 2 of the LCD module to the output of the first comparator, the clock starts when the user's vocal intensity exceeds the threshold. Thus the clock displays the elapsed time that the user is screaming. A second clock module with pin 2 connected to main power displays the elapsed time that the device is on. Thus a parent could tell a child that he'll earn a reward if his elapsed time on the first clock is less than one hour. The second clock shows the parent that the child didn't cheat by switching the device off and on to reset the first clock.

Thus, by utilizing the above construction, a device for monitoring vocal intensity is realized.

It will thus be seen that the objects set forth above, among those made apparent from the preceding description, are efficiently attained and, since certain changes may be made in the above constructions without departing from the spirit and scope of the invention, it is intended that all matter contained in the above description or shown in the accompanying drawings shall be interpreted as illustrative, and not in a limiting sense.

It will also be understood that the following claims are intended to cover all of the generic and specific features of the invention, herein described, and all statements of the scope of the invention which, as a matter of language, might be said to fall therebetween.

What is claimed is:

1. A device that monitors a user's vocal intensity and provides such information in a format selected from the group consisting of tactile, auditory, and temporal information presentation.

2. A device according to claim 1, in which said tactile presentation uses a vibrating motor.

3. A device according to claim 1, in which said audible presentation is one or more synthesized tones.

4. A device according to claim 1, in which said audible presentation consists of a reproduction of said user's voice.

5. A device according to claim 1, in which said temporal presentation consists of a clock displaying elapsed time indicating a relationship between said user's vocal intensity and a threshold.

6. A device according to claim 1, in which said vocal intensity is analyzed in accordance with parameters selected from the group consisting of vocal volume or amplitude, and vocal frequency or pitch.

7. A device that monitors a user's vocal intensity and induces speech motor changes in said user.

8. A device according to claim 7, in which said speech motor changes are induced by altering the frequency or pitch at which said user hears his or her voice.

9. A method of speech training comprising a device that monitors a user's vocal intensity and provides such information in a format selected from the group consisting of tactile, auditory, and temporal information presentation.

10. The method of claim 9, for treating vocal nodules.

11. The method of claim 9, for treating speech disorders associated with Parkinson's disease.

12. The method of claim 9, for treating stuttering.
